(19) United States
(12) Patent Application Publication
(10) Pub. No.: US 2026/0157943 A1
(43) Pub. Date: Jun. 11, 2026

(54) COMPOSITION FOR FOUNDATION IN SPRAY FORM

(71) Applicant: QUADRA GROUP S.R.L., MILAN (MI) (IT)

(72) Inventor: FABIO DURIGHEL, MILAN (MI) (IT)

(21) Appl. No.: 18/708,007

(22) PCT Filed: Nov. 8, 2022
(86) PCT No.: PCT/IB2022/060730

§ 371 Date: May 7, 2024

(30) Foreign Application Priority Data
Nov. 8, 2021 (IT) 102021000028394

(51) Int. Cl. A61K 8/04; A61K 8/31; A61K 8/64; A61Q 1/12

(52) U.S. Cl. CPC A61K 8/046; A61K 8/31; A61K 8/64; A61Q 1/12; A61K 2800/43; A61K 2800/88

(57) ABSTRACT

Cosmetic compositions and specifically foundation compositions in a spray form.

### TECHNICAL FIELD

The present invention relates to cosmetic compositions and in particular relates to foundation compositions in a spray form, or capable of being made in a spray or sprayable form.

### TECHNICAL BACKGROUND

The cosmetics industry is a constantly developing industry that must continually satisfy a demanding public that demands better and better performing products.

Foundations are cosmetic products used to even out the complexion, cover blemishes, and sometimes change the natural skin tone. They come in a variety of forms including emulsions, compact creams, sticks, and pressed or free powders.

A foundation composition is generally composed of numerous ingredients. The main ingredients are covering pigments and coloring pigments. In addition, there are filler ingredients, fragrances, and, depending on the type of composition, emulsion ingredients, preservatives, and/or antioxidants.

Foundation compositions are complex compositions in that they contain many ingredients and components having different chemical and physical natures, and because of this, foundation compositions can also be very difficult to formulate to give a stable, high-performance final product. Foundation compositions can also give rise to stability problems over time, separation phenomena between the various ingredients or components that compose it, or problems with poor hold after application to the skin. In addition, foundation compositions are often formulated with synthetic or extracted chemical ingredients that may contain traces of heavy metals, metal catalysts, and organic extraction solvents that can give rise to skin irritation or allergy and thus be not beneficial either to the body or to the environment.

Another issue strongly felt by operators in the sector with reference to the current foundation compositions available on the market is the difficulty of applying them by the end user because existing foundation compositions are often too liquid or too compact and dense, and an even and ideal application cannot be achieved if the foundation compositions are not applied by an experienced hand.

Therefore, there is a need for foundation products that give a natural complexion, offer long staying power, and are quick and easy to apply.

Foundation products are also required to cover any hyper-pigmentation of the skin, (e.g., sun or hormonal spots) thus saving the end user from having to first apply a layer of concealer.

Finally, there is a felt need for operators in the sector to have foundation products that not only allow coverage of such spots but at the same time act effectively to reduce the spot itself.

To date, there are no foundation products on the market that meet the above requirements, and it is on these aspects that the Applicant has focused with its continuous research and development. The Applicant, in response to the above requirements, has developed an innovative foundation composition that allows for a natural complexion. The compositions developed by the Applicant are also long-lasting compositions that allow skin spots to be covered, while at the same time allowing them to be significantly reduced.

In addition, the foundation compositions developed by the Applicant are easy to apply. In particular, ease of application is favored by the spray/aerosol formulation that results in the formation of droplets with sizes in the micron range, e.g., of average particle size (mean particle diameter) ranging from 0.001 μm to 100 μm, which promotes better product distribution uniformity. It is well known that a spray/aerosol system is a colloidal system consisting of liquid or solid particles, usually ranging in size from 15 μm to 50 μm, dispersed in a gas. It is formed by mechanical dispersion of a liquid, for example under the action of a propellant gas or by bubbling air or other gases into liquid containers.

Cosmetic compositions according to the present invention also can be used by all categories of female and male subjects.

Finally, the compositions according to the present invention are easy to prepare and cost-effective.

These purposes and others, which will become clear from the detailed description that follows, are achieved by the compositions of the present invention through the technical features claimed in the united claims.

### SUMMARY OF THE INVENTION

The present invention relates to a foundation composition comprising:
(a) Silicone-based microparticles, and
(b) an oligopeptide consisting of a number of amino acids from 7 to 16, wherein said amino acids are selected from the group consisting of: arginine, aspartic acid, isoleucine, glutamine, leucine, serine, threonine, and tryptophan,
   wherein said (a) silicone-based microparticles comprise or, alternatively, consist of a mixture comprising or, alternatively, consisting of:
a cross-linked polysiloxane;
polymethylsesquioxane;
phenoxyethanol;
ethylhexylglycerin; and
polyoxyethers of lauryl alcohol;
   preferably, said (a) silicone-based microparticles comprise or, alternatively, consist of a mixture (M1) comprising or, alternatively, consisting of: polysilicone-11, polymethylsesquioxane, HDI/trimethylol hexyllactone crosspolymer, Laureth-12, phenoxyethanol and ethylhexylglycerin, preferably said mixture is an aqueous mixture or emulsion.

The foundation composition takes the form of an emulsion. Once the foundation composition is introduced into a container, such as a spray can, and added with a pressurized gas such as, for example, a propellant gas known to the person skilled in the art, a foundation composition is obtained in a spray or sprayable form, which is also an object of the present invention.

Preferably, said mixture is in an emulsion form, such as an aqueous emulsion.

The mixture is a combination of an elastomer, polysilicone-11 (in powder form) and other ingredients, some of them also in powder form. In the presence of water, said mixture appears as an aqueous emulsion of silicone-based microparticles. For example, 1% by weight of the mixture in water has a pH from 4 to 7. Preferably, the mixture is used within the foundation composition FC in an amount by weight from 1% to 20%, preferably from 3% to 9%, e.g. 4%, or 5%, or 6%, or 7%, or 8%.

The present invention also relates to a kit comprising:
the composition according to the present invention; and
a brush.

### DETAILED DESCRIPTION OF THE INVENTION

The present invention relates to cosmetic foundation compositions. Specifically, it relates to foundation compositions in a spray form, or capable of being made in a spray or sprayable form.

The present invention also relates to the use of said compositions to improve the appearance of the skin, even out the complexion and reduce any spots (hyper-pigmentation) present on the skin.

The compositions according to the present invention are for topical cosmetic use and effectively improve the outward appearance of the skin, allowing any defects to be covered and giving an even complexion.

Advantageously, compositions according to the present invention can be spray compositions, or capable of being made in a spray or sprayable form.

Therefore it is an object of the present invention, a foundation composition comprising:
(a) silicone-based microparticles;
(b) an oligopeptide consisting of a number of amino acids from 7 to 16, wherein said amino acids are selected from the group consisting of: arginine, aspartic acid, isoleucine, glutamine, leucine, serine, threonine, and tryptophan;
   and wherein said silicone-based microparticles comprise a mixture comprising or, alternatively, consisting of:
a cross-linked polysiloxane;
polymethylsesquioxane;
phenoxyethanol;
ethylhexylglycerin; and
polyoxyethers of lauryl alcohol.

Preferably, said mixture may comprise or, alternatively, consist of:
polysilicone-11 (INCI Name, CAS 156065 Feb. 2, 63394 Feb. 5) from 50% to 70%, e.g., from 59% to 62%;
water, (CAS 7732-18-5) from 30% to 50%, e.g., from 36% to 39%,
Laureth-12 (INCI Name, CAS 9002-92-0) from 0.5% to 2%, e.g., from 0.8% to 1.4%;
phenoxyethanol (INCI Name, CAS 122-99-6) from 0.5% to 1.5%, e.g., from 0.7% to 0.9%;
HDI/trimethylol hexyl-lactone crosspolymer (INCI Name, CAS 9009-54-5, 53504-41-9, 129757-76-2) from 0.001% to 1%, e.g., from 0.01% to 0.9%;
ethylhexylglycerin (INCI Name, CAS 70445-33-9) from 0.01% to 0.3%, e.g., from 0.05% to 0.15%;
polymethylsesquioxane (INCI Name, CAS 68554-70-1) from 0.001% to 1%, e.g., from 0.01% to 0.9%.

Said cross-linked polysiloxane, it is a polymer molecule containing siloxane bonds in the main chain (—O—Si—O) and may contain functional groups that allow crosslinking.

The polysiloxanes that can be used in the present context are polysiloxanes typically used in cosmetic products. For example, a polysiloxane-11 (polysilicone-11) can be used, which is a synthetic siloxane generally used in cosmetic formulations.

Polymethylsesquioxane in the present context refers to a polymer obtainable by hydrolytic condensation of a trifunctional silane such as methyltrichlorosilane. The polymethylsesquioxane that can be used in the present context is commercially available.

Phenoxyethanol in the present context means an organic compound having formula C₆H₅OC₂H₄OH.

Ethylhexyglycerin, or octoxyglycerin, in the present context refers to an alkyl ether of glycerin that can be represented by the following formula: C₁₁H₂₄ O₃.

The polyoxyethers of lauryl alcohol used in the present context may be compounds such as the “Laureth-n” series (e.g. Laureth-1, Laureth-2, Laureth-3, Laureth-5, Laureth-6, Laureth-7, Laureth-8, Laureth-9, Laureth-10, Laureth-11, Laureth-12, etc.). The number “n” in the name indicates the average number of ethylene oxide units in the molecule. Preferably, Laureth-12 is used.

Surprisingly, the Applicant found that the addition of said silicone-based microparticles to the foundation composition results in a unifying and glowing effect. Furthermore, the addition of said microparticles reduces signs of aging such as wrinkles and skin blemishes.

The mixture used to prepare said silicone-based microparticles may also contain one or more cross-linked Trimethylol hexylactone polymers.

Said microparticles can have a diameter ranging from 0.5 μm to 50 μm, preferably from 1 μm to 15 μm, for example, from 1 μm to 6 μm.

Said microparticles can be dispersed in water to form a milky solution that can be added to the final foundation composition. Alternatively, said microparticles can be added in the form of a solid powder and mixed into the foundation composition.

Preferably said microparticles may comprise or, alternatively, consist of a mixture of:
polyoxysilicone-11; and
water; and
polymethylsilsesquioxane; and
HDI/Trimethylol; and
cross-linked hexyllactone; and
laureth-12; and
phenoxyethanol; and
ethylhexiglycerin.

In an embodiment, said silicone-based microparticles can be commercially purchased, for example they may be Gransil blur-X11 microparticles, manufactured by Grant Industries Inc.

Advantageously, said microparticles are present in a weight concentration with respect to the final formulation ranging from 1% to 20%, preferably from 3% to 9%, e.g., 5%.

In addition, the foundation composition according to the present invention comprises an oligopeptide consisting of a number of amino acids from 7 to 16, wherein said amino acids are selected from the group consisting of: arginine, aspartic acid, isoleucine, glutamine, leucine, serine, threonine, and tryptophan.

Preferably, said oligopeptide is, for example, an oligopeptide consisting of 13 amino acids, said amino acids being selected from the group consisting of: arginine, aspartic acid, isoleucine, glutamine, leucine, serine, threonine, and tryptophan.

Said oligopeptide used in the present context can be commercially purchased. For example, it may be an oligopeptide-34 which is a peptide that consists of 13 amino acids selected from the group consisting of: arginine, aspartic acid, isoleucine, glutamine, leucine, serine, threonine, and tryptophan.

This oligopeptide can be purchased commercially, for example, from Caregen Ltd.

Said oligopeptide, preferably said oligopeptide consisting of 13 amino acids, has an average molecular weight ranging from 1300 Da to 1600 Da, preferably ranging from 1400 Da to 1500 Da, e.g., 1460-1480 Da, and may be present in the foundation composition as, for example, the product CG-TGP2-oligopeptide -34 encapsulated solution, in the form of an aqueous solution.

Said oligopeptide, preferably said oligopeptide consisting of 13 amino acids, for example, in aqueous solution, may comprise:
water: as required to 100%, CAS 7732-18-5;
hydrogenated lecithin: from 0.05% to 2%, e.g., 0.1%, CAS 92128-87-5;
sodium oleate: from 0.001% to 0.1%, e.g., 0.01%, CAS 143-19-1;
glycine from soybean oil: from 0.01% to 0.5%, e.g., 0.1%, CAS 8001-22-7;
glycerin: from 0.1% to 1.5%, e.g. 1%, CAS 56-81-5;
disodium-EDTA, from 0.01% to 0.05, e.g. 0.03%, CAS 139-33-3;
phenoxyethanol, from 0.5% to 1.5%, e.g. 1%, CAS 122-99-6;
oligopeptide-34: from 0.01% to 0.1%, e.g., 0.05%.

Said oligopeptide, preferably said oligopeptide consisting of 13 amino acids, when present as an aqueous solution is present in the foundation composition in an amount ranging from 0.5% to 5%, preferably from 1% to 4%, e.g. 2%, or 2.5%, or 3%, or 3.5%.

The Applicant surprisingly found that the addition of this peptide, preferably said oligopeptide, to a foundation composition can reduce the appearance of hyper-pigmentation of the skin.

Hyper-pigmentations or skin spots present as an alteration of the normal skin tone with small dark patches ranging in color from brown to black. They are generally caused by excessive production of melanin, the pigment that gives color to skin and hair. They can be determined by several factors, including excessive sun exposure, hormonal problems, or skin aging. They are common in fair skin, and often occur due to a number of concomitant causes, such as sun exposure during a hormonally sensitive period such as pregnancy or menopause.

They are often treated using specific products that must be applied overnight, or covered with specific concealers.

However, the Applicant surprisingly found that the addition of an oligopeptide, as described in the present context, to the foundation formulation allows for a reduction in sunspots, and also allows for an application advantage due to the simultaneous coverage of spots by the action of the foundation.

Advantageously, the foundation composition according to the present invention comprises said oligopeptide in a concentration by weight % ranging from 0.0001% to 1%, preferably from 0.0005% to 0.5%, even more preferably from 0.001% to 0.09%, e.g., 0.05%.

Advantageously, the foundation composition according to the present invention is in a spray form, or capable of being made in a spray or sprayable form.

The composition formulated to optimize the spraying of the product allows for rapid application without the product dripping or clumping. In particular, the composition according to the present invention once sprayed has a light, powder-like consistency that results in easy application for the end user.

When formulated to be sprayed, the composition according to the present invention may contain a C₂-C₄ alkane, for example, ethane, propane, butane, iso-butane as propellant agents. The compositions may comprise C₂ to C₄ alkanes in an amount by weight % ranging from 20% to 75%, preferably ranging from 25% to 70%, even more preferably ranging from 40% to 69% with respect to the total weight of the composition, e.g. 68%.

Preferably, as a propellant agent the composition according to the present invention comprises at least one alkane selected from the group consisting of propane, butane, isobutane and mixtures thereof.

Titanium dioxide can be used as covering pigment. This compound produces a white color that can disperse light, and enhance the natural color of the skin.

Preferably, titanium dioxide is used as the covering pigment in the present invention.

Said covering pigments can be included in an amount by weight % ranging from 0.5% to 10%, preferably in an amount ranging from 1% to 6%, e.g. at 4.27%.

Red, yellow and black iron oxides can be used as color pigments, which are appropriately mixed to create color shades that match different skin types.

Said color pigments can be included in an amount by weight % ranging from 0.1% to 5%, preferably in an amount ranging from 0.2% to 3%, e.g. in an amount of 0.7%, 1.3%, 2.6%.

In a preferred embodiment, the ratio of yellow iron oxide (Y), red iron oxide (R) and black iron oxide (B) (Y:R:B) is 1:1:1.

In a preferred embodiment, the ratio of yellow iron oxide (Y), red iron oxide (R) and black iron oxide (B) (Y:R:B) is 4:1:1.

In a preferred embodiment, the ratio of yellow iron oxide (Y), red iron oxide (R) and black iron oxide (B) (Y:R:B) is 3:3:1.

In a preferred embodiment, the ratio of yellow iron oxide (Y), red iron oxide (R) and black iron oxide (B) (Y:R:B) is 6.5:2:1.

Compositions according to the present invention can be prepared using one or more solvents approved for cosmetic preparations. For example, in an embodiment, the solvent may be selected from the group comprising or, alternatively, consisting of: water, glycerol, propylene glycol, butylene glycol, pentylene glycol, ethoxylated or propoxylated diglycols, ethanol, propanol, isopropanol, and mixtures thereof.

Preferably, a mixture of water and pentylene glycol is used as a solvent.

The ratio of water to pentylene glycol, used as solvents, can range from 7:1 to 5:1, such as 6:1.

Compositions according to the present invention may also comprise technological additives commonly used in cosmetics.

For example, compositions according to the present invention may comprise, surfactants, emulsifiers, thickeners, rheological additives, stabilizing agents, antioxidants, preservatives, fragrance oils/fragrances, emollient agents, filling agents, antimicrobial agents, chelating agents, and/or UV filters.

The foundation composition according to the present invention may comprise silicone compounds, such as dimethylpolysiloxanes, methylphenylpolysiloxanes, and cyclic silicones, such as cyclopentasiloxanes. Also included may be alkyl-methicones (methicone) with an average chain length from 16 to 6 units, preferably ranging from 12 to 9, e.g., 8. These compounds act as emollient, and conditioning agents for the skin.

Preferably, the composition according to the present invention comprises an emollient agent selected from the group comprising or, alternatively, consisting of cyclopentasiloxane, caprylic triglyceride, soybean oil (Glycine soja oil) and mixtures thereof.

Said emollient may be included in an amount by weight % with respect to the total weight of the composition ranging from 5% to 20%, preferably ranging from 10% to 15%, such as an amount of 15% by weight.

The foundation composition according to the present invention may also comprise filler/filling agents such as talc, mica, sericite, or mixtures thereof. The use of these kinds of filling agents can help spread the primary coverage pigments on the face. They also make the product easier to use.

Preferably, talcum powder is used as the filler/filling agent in the present invention.

Said filler/filling agent may be included in an amount by weight % with respect to the total weight of the composition ranging from 1% to 20%, preferably ranging from 2% to 15%, e.g., in an amount of 10% by weight.

In addition, compositions according to the present invention can contain fragrances commonly used for cosmetic products. A preferred example of a fragrance that can be used is vanilla fragrance, and/or almond blossom fragrance. These types of fragrances, increase the pleasantness of application of the composition according to the present invention.

In a preferred embodiment, the perfurming fragrance is selected from the group comprising or, alternatively, consisting of: vanilla essence, almond blossom, limonene, glycerin, and mixtures thereof.

Said fragrance may be included in an amount by weight % with respect to the total weight of the mixture ranging from 0.05% to 0.5%, preferably from 0.1% to 0.4%.

Selected thickeners selected from the group comprising or, alternatively, consisting of: disteardimonium hectorite, hydroxyethyl cellulose, guar gum, gelatin, magnesium aluminum silicate, silica, bentonite, or propylene carbonate (PC) may be used as rheology modifiers.

Preferably, said additive/rheological modifier can be selected from the group consisting of: disteardimonium hectorite (disteardimonium hectorite) (DH), propylene carbonate (PC), and mixtures thereof.

Said rheological additive may be included in an amount by weight % with respect to the total weight of the composition ranging from 0.1% to 1%, preferably ranging from 0.2% to 0.9%, e.g., 0.7%.

Compositions according to the present invention may contain emulsifying agents commonly used in cosmetics.

Preferably, said emulsifying agent is selected from the group comprising or, alternatively, consisting of cetyl PEG/PPG-10/1 dimethicone, hydrogenated lecithin, sodium oleate and mixtures thereof.

The latter are commercially available chemical compounds.

Said emulsifying agent may be included in an amount by weight % with respect to the total weight of the composition ranging from 0.1% to 1.5%, preferably from 0.2% to 0.9%.

The composition according to the present invention may also comprise UV photoprotective agents (UVA and/or UVB). For, example, as photoprotective agents, the composition according to the present invention may comprise organic substances (photoprotective filters) that are liquid or crystalline at room temperature and that are capable of absorbing ultraviolet radiation, and giving back the absorbed energy again in the form of longer wavelength radiation, such as heat.

Preferably, ethylhexyl methoxycinnate is used in the composition according to the present invention as the photoprotective agent for UVA and/or UVB.

Said photoprotective agent may be included in an amount by weight % with respect to the weight of the total composition ranging from 0.2% to 1.5%, preferably ranging from 0.3% to 0.9%.

The composition according to the present invention may also comprise antioxidant agents. Substances such as tocopherol, tocopherol acetate, tocopherol palmitate, ascorbic acid, and vitamin complexes may be used as antioxidant agents.

Preferably, in the composition according to the present invention, tocopherol acetate is used as the antioxidant agent.

Said antioxidant agent may be included in an amount by weight % with respect to the total weight of the composition ranging from 0.05% to 0.9%, preferably from 0.08% to 0.4%, e.g., 0.1%.

The composition according to the present invention may also comprise chelating agents, preservatives, and/or anti-microbial agents.

Preferably, disodium EDTA is used as the chelating agent in the composition according to the present invention.

Said chelating agent may be included in an amount by weight % with respect to the total weight of the composition ranging from 0.005% to 0.1%, preferably ranging from 0.008% to 0.09%, e.g., 0.05%.

The composition according to the present invention may also comprise preservative agents commonly used in the industry.

As anti-microbial agents in the composition can be use, all substances effective against gram-positive bacteria, such as, for example, 4-hydroxybenzoic acid and salts and esters thereof, N-(4-chlorophenyl)-N′-(3, 4-dichlorophenyl) urea, 2,4,4′-trichloro-2′-hydroxydiphenyl ether (triclosan), 4-chloro-3,5-dimethylphenol, benzyl benzoate.

Preferably, in the composition according to the present invention, benzyl benzoate is used as the anti-microbial agent.

Said anti-microbial agent may be included in an amount by weight % with respect to the total weight of the composition ranging from 0.005% to 0.02%, preferably ranging from 0.008% to 0.01%.

### Spray Composition Kit and Brush

The present invention also relates to a KIT comprising.
The foundation cosmetic composition, preferably in a spray form;
A brush.

The application of the composition according to the present invention with the help of the brush according to the present invention is particularly effective for even application, and for depositing the product in the areas covered by spots, especially in delicate areas such as the upper lip area.

### EMBODIMENTS

Preferred embodiments FRn of the present invention are given below.

FR1. A foundation composition comprising:
(c) silicone-based microparticles,
(d) an oligopeptide consisting of a number of amino acids from 7 to 16, wherein said amino acids are selected from the group consisting of: arginine, aspartic acid, isoleucine, glutamine, leucine, serine, threonine, and tryptophan, wherein said silicone-based microparticles comprise or, alternatively, consist of a mixture comprising or, alternatively, consisting of:
a cross-linked polysiloxane;
polymethylsesquioxane;
phenoxyethanol;
ethylhexylglycerin; and
polyoxyethers of lauryl alcohol.

FR2. The composition according to FR1, wherein said oligopeptide consists of 13 amino acids selected from the group consisting of: arginine, aspartic acid, isoleucine, glutamine, leucine, serine, threonine, and tryptophan.

FR3. The composition according to any one of FR1-FR2, wherein said composition is in a liquid or spray form.

FR4. The composition according to FR3, wherein said composition additionally comprises a propellant agent selected from the group comprising or, alternatively, consisting of propane, butane, isobutane, and mixtures thereof.

FR5. The composition according to any one of FR1-FR4, wherein said oligopeptide is in a concentration ranging from 0.001% to 1% by weight.

FR6. The composition according to any one of the preceding FRs, wherein said microparticles are contained in an amount from ranging from 0.5 μm to 50 μm.

FR7. The composition according to any one of the preceding FRs, wherein said composition additionally comprises a coloring compound selected from the group comprising or, alternatively, consisting of yellow iron oxide, red iron oxide, black iron oxide, and mixtures thereof.

FR8. The composition according to any one of the preceding FRs, wherein said composition additionally comprises a titanium dioxide, preferably said titanium dioxide being in an amount by weight % ranging from 0.5% to 5%, preferably in an amount ranging from 1% to 3%, e.g. at 2%.

FR9. The composition according to any one of the preceding FRs, wherein said composition additionally comprises:
a filling agent selected from the group comprising or, alternatively, consisting of: talc, mica, sericite, or mixtures thereof;
a perfuming fragrance selected from the group comprising or, alternatively, consisting of: vanilla essence, almond blossom, limonene, glycerin, and mixtures thereof;
a rheological additive selected from the group consisting of: disteardimonium hectorite (DH), propylene carbonate (PC), and mixtures thereof;
an emulsifying agent selected from the group consisting of cetyl peg/PPG-10/1 dimethicone, hydrogenated lecithin, sodium oleate, and mixtures thereof;
an antioxidant agent selected from the group comprising or, alternatively, consisting of tocopherol, tocopherol acetate, tocopherol palmitate, ascorbic acid, and vitamin complexes, preferably said antioxidant agent is tocopherol acetate.

FR10. A kit comprising:
the composition according to any one of the preceding FRs; and
a brush.

### Experimental Part

Further details of the invention are illustrated in the following Example.

The compositions are tested on a female subject with skin spots.

### Example 1

TABLE 1
COMPOSITION 1

| | | |
|---|---|---|
| | | % WEIGHT/ |
| INGREDIENT | FUNCTION | WEIGHT |

| | |
|---|---|
| Butane | propellant |
| Titanium dioxide | Pigment filler |
| Yellow pigment | Iron Oxide CI77492 |
| Red pigment | Iron Oxide CI77491 |
| Black pigment | Iron oxide CI 77499 |
| Oligopeptide-34 | |
| Silicone-based microparticles | Gransil blur X11 |
| solvent | Water:pentylene glycol |
| | 4:1 |

What is claimed is:

1. A foundation composition comprising:
(a) silicone-based microparticles,
(b) an oligopeptide consisting of a number of amino acids from 7 to 16, wherein said amino acids are selected from the group consisting of arginine, aspartic acid, isoleucine, glutamine, leucine, serine, threonine, and tryptophan,
wherein said silicone-based microparticles comprise a mixture comprising:
polysilicone-11, polymethylsesquioxane, HDI/trimethylol hexyl-lactone crosspolymer, Laureth-12, phenoxyethanol and ethylhexylglycerin.

2. The composition according to claim 1, wherein said mixture may comprises:
polysilicone-11 (INCI Name, CAS 156065 Feb. 2, 63394 Feb. 5) from 50 to 70%;
water (CAS 7732-18-5) from 30% to 50%, preferably from 36% to 39%;
Laureth-12 (INCI Name, CAS 9002-92-0) from 0.5% to 2%;
phenoxyethanol (INCI Name, CAS 122-99-6) from 0.5% to 1.5%;
HDI/trimethylol hexyl-lactone crosspolymer (INCI Name, CAS 9009-54-5, 53504-41-9, 129757-76-2) from 0.001% to 1%;
ethylhexylglycerin (INCI Name, CAS 70445-33-9) from 0.01% to 0.3%, preferably from 0.05% to 0.15%;
polymethylsesquioxane (INCI Name, CAS 68554-70-1) from 0.001% to 1.

3. The composition according to claim 1, wherein said oligopeptide consists of 13 amino acids selected from the group consisting of: arginine, aspartic acid, isoleucine, glutamine, leucine, serine, threonine, and tryptophan.

4. The composition according to claim 3, wherein said oligopeptide is present in aqueous solution, and may comprises:
water: as required to 100%, CAS 7732-18-5;
hydrogenated lecithin: from 0.05% to 2%, CAS 92128-87-5;
sodium oleate: from 0.001% to 0.1% CAS 143-19-1;
glycine from soybean oil: from 0.01% to 0.5%, CAS 8001-22-7;
glycerin: from 0.1% to 1.5%, CAS 56-81-5;
disodium-EDTA, from 0.01% to 0.05, CAS 139-33-3;
phenoxyethanol, from 0.5% to 1.5%, CAS 122-99-6;
oligopeptide-34: from 0.01% to 0.1%.

5. The composition according to claim 1, wherein said composition is in a liquid or spray form.

6. The composition according to claim 5, wherein said composition further comprises a propellant agent selected from the group comprising propane, butane, isobutane, and mixtures thereof.

7. The composition according to claim 1, wherein said oligopeptide is in a concentration ranging from 0.001% to 1% by weight.

8. The composition according to claim 1, wherein said microparticles have a diameter of from 0.5 μm to 50 μm.

9. The composition according to claim 1, wherein said composition further comprises a coloring compound selected from the group consisting of yellow iron oxide, red iron oxide, black iron oxide, and mixtures thereof.

10. The composition according to claim 1, wherein said composition further comprises a titanium dioxide in an amount by weight % ranging from 0.5% to 5%.

11. The composition according to claim 1, wherein said composition further comprises:
a filling agent selected from the group consisting of talc, mica, sericite, and mixtures thereof;
a perfuming fragrance selected from the group consisting of vanilla essence, almond blossom, limonene, glycerin, and mixtures thereof;
a rheological additive selected from the group consisting of disteardimonium hectorite (DH), propylene carbonate (PC), and mixtures thereof;
an emulsifying agent selected from the group consisting of cetyl peg/PPG-10/1 dimethicone, hydrogenated lecithin, sodium oleate, and mixtures thereof;
an antioxidant agent selected from the group consisting of tocopherol, tocopherol acetate, tocopherol palmitate, ascorbic acid, and vitamin complexes, preferably said antioxidant agent is tocopherol acetate.

12. A kit comprising:
the composition according to claim 1; and
a brush.

13. The composition according to claim 2, wherein said mixture comprises:
59% to 62% polysilicone-11;
36% to 39% water;
0.8% to 1.4% Laureth-12;
0.7% to 0.9% phenoxyethanol;
0.01% to 0.9% HDI/trimethylol hexyl-lactone crosspolymer;
0.05% to 0.15% ethylhexylglycerin;
0.01% to 0.9% polymethylsesquioxane.

14. The composition according to claim 4, wherein said oligopeptide comprises:
water: as required to 100%;
0.1% hydrogenated lecithin;
0.01% sodium oleate;
0.1% glycine from soybean oil;
1% glycerin;
0.3% disodium-EDTA;
1% phenoxyethanol;
0.05% oligopeptide-34.

15. The composition according to claim 10, wherein said titanium dioxide is present in an amount by weight % ranging from 1% to 3%.
